(19) United States
(12) Patent Application Publication
(10) Pub. No.: US 2022/0331275 A1
(43) Pub. Date: Oct. 20, 2022

(54) COMBINATION TREATMENTS FOR CENTRAL NERVOUS SYSTEM DISORDERS

(71) Applicant: PhilERA New Zealand Ltd., Auckland (NZ)

(72) Inventor: Nicholas SISTI, Mineola, NY (US)

(21) Appl. No.: 17/619,571

(22) PCT Filed: Jun. 17, 2020
(86) PCT No.: PCT/US2020/038131

§ 371 Date: Dec. 15, 2021

Related U.S. Application Data
(60) Provisional application No. 62/862,415, filed on Jun. 17, 2019.

(51) Int. Cl. A61K 31/197; A61K 31/132; A61P 25/28

(52) U.S. Cl. CPC A61K 31/197; A61K 31/132; A61P 25/28

(57) ABSTRACT

The inventions relate to compositions and methods for treating or preventing disorders of the central nervous system comprising administering to a patient, separately or in combination, an agent capable of normalizing copper levels and/or values and an agent capable of normalizing vitamin B₅ levels, including in Alzheimer's disease patients.

### RELATED APPLICATIONS

This application is a 371 Application of International Application No. PCT/US2020/038131, filed Jun. 17, 2020, which claims the benefit of priority to U.S. Provisional Application Ser. No. 62/862,415, filed Jun. 17, 2019, which are incorporated by reference in their entirety.

### INCORPORATION BY REFERENCE

All patents, patent applications and other documents listed herein are hereby incorporated by reference in their entirety.

### FIELD

This invention concerns methods of treatment, prevention or amelioration of central nervous system and other disorders.

### BACKGROUND

The following description includes information that may be useful in understanding the present invention. It is not an admission that any of the information provided herein is prior art, or relevant, to the presently described or claimed inventions, or that any publication or document that is specifically or implicitly referenced is prior art or a reference that may be used in evaluating patentability of the described or claimed inventions.

Certain copper chelators have been described for use in treating certain disorders, including cardiovascular, glucose and vascular disorders. U.S. Pat. No. 10,543,178 describes use of a succinic acid addition salt of triethylenetetramine in a method of treating subjects for diabetic neuropathy. Related U.S. Pat. No. 9,993,443 describes use of a succinic acid addition salt of triethylenetetramine in a method of treating subjects for tissue damage associated with a number of cardiac, glucose related and vascular disorders: (a) diabetic cardiomyopathy, diabetic acute coronary syndrome, diabetic hypertensive cardiomyopathy, acute coronary syndrome associated with impaired glucose tolerance, acute coronary syndrome associated with impaired fasting glucose, hypertensive cardiomyopathy associated with impaired glucose tolerance, hypertensive cardiomyopathy associated with impaired fasting glucose, ischemic cardiomyopathy associated with impaired glucose tolerance, or ischemic cardiomyopathy associated with impaired fasting glucose; (b) myocardial infarction, ischemic cardiomyopathy associated with coronary heart disease, cardiomyopathy, myocarditis, idiopathic cardiomyopathy, metabolic cardiomyopathy, alcoholic cardiomyopathy, drug-induced cardiomyopathy, ischemic cardiomyopathy, and hypertensive cardiomyopathy, acute coronary syndrome not associated with any abnormality of glucose metabolism, hypertensive cardiomyopathy not associated with any abnormality of glucose metabolism, or ischemic cardiomyopathy not associated with any abnormality of glucose metabolism; or (c) disease states of an artery selected from aorta, carotid, cerebrovascular, coronary, renal, retinal, iliac, femoral, popliteal, vasa nervorum, arteriolar tree, and capillary bed, and atheromatous disorders of the major blood vessels selected from the aorta, the coronary arteries, the carotid arteries, the cerebrovascular arteries, the renal arteries, the iliac arteries, the femoral arteries, or the popliteal arteries.

The use of various chelators to lower copper (II) values in patients with tissue damage in myocardial tissue, kidney tissue, eye tissue, nerve tissue, and vascular tissue is described in U.S. Pat. No. 8,987,244, including trientine, 2,2,2 tetramine tetrahydrochloride (TETA) and 2,3,2 tetramine tetrahydrochloride.

U.S. Pat. No. 8,563,538 describes the use of 2,3,2 tetramine compositions in methods of treating heart failure in a non-diabetic human subject, including 2,3,2 tetramine hydrochloride salts, e.g., 2,3,2 tetramine tetrahydrochloride. Doses include 0.1 mg/kg, 1.0 mg/kg, 10 mg/kg, 100 mg/kg, and dose ranges from 0.001-100 mg/kg/day, from 0.01-25 mg/kg/day, from 0.05-10 mg/kg/day, from 0.1-5 mg/kg/day, and from 0.5-5 mg/kg/day. Administration of 2,3,2 tetramine in amounts ranging from 1-1000 milligrams from one to four times per day, for example, orally, in a tablet or capsule, is also described. In one embodiment, the composition includes a zinc salt. U.S. Pat. No. 8,034,799 also describes and claims methods of treating heart failure in a non-diabetic human subject with an agent capable of reducing copper levels, for example, copper (II), including copper chelators such as trientine, as well as 2,3,2 tetramine, D-penicillamine, N-acetylpenicillamine, trithimolybdate, and tetrathimolybdate. It also describes and claims the use of 2,2,2 tetramine and 2,3,2 tetramine hydrochloride salts.

U.S. Pat. No. 7,928,094 describes the use of the copper chelator triethylenetetramine dihydrochloride (also known as trienes or trien-2HCl or trientine dihydrochloride) in treating a human for one or more conditions associated with long-term complications of diabetes in an amount ranging from about 9-200 mg/kg per day. Conditions claimed include cardiomyopathy, atherosclerosis, renal complications, nephropathy, deterioration of the eyesight, retinopathy, cataract formation, and neuropathy. In one embodiment, the trienes can be given in an amount ranging from about 1.2 to about 2.4 grams per day. The patent also claims the use of trienes for ameliorating tissue damage in humans associated with diabetes, including tissue damage in cardiac muscle, cardiac microvasculature, brain, brain microvasculature, kidney, kidney microvasculature, skeletal muscle, skeletal muscle microvasculature, skin tissue, skin tissue, microvasculature, retinal tissue, retinal microvasculature, peripheral nerve tissue, and peripheral nerve microvasculature. Administration of oral and long-release forms of the triene is described.

It is understood that Central Nervous System (CNS) disorders are in need of new therapies. CNS diseases broadly include diseases, disorders and conditions of any component of the brain and the spinal cord. They include disorders in which the nervous system is affected during the progression of the diseases such as neurodegenerative diseases (e.g., Alzheimer's disease, Parkinson's disease, amyotrophic lateral sclerosis (ALS), degenerative ataxias such as Friedrich's ataxia, multiple sclerosis, multiple system atrophy and leukodystrophies), cerebrovascular diseases, seizures, epilepsy, viral diseases (e.g., meningitis, encephalitis), brain tumors and neuroinflammatory diseases. CNS disorders also include disorders in which the nervous system is only affected during the latest stages of the development of the disorder. The disorders comprise rare metabolic diseases such as organic acidemias or fatty acid disorders and genetic mitochondrial disorders.

Neurodegenerative diseases are characterized by the progressive loss of structure or function of neurons, including death of neurons. These conditions are progressive and often fatal. The process of neurodegeneration is not well understood and the diseases that stem from it have, as yet, no cures in spite of treatments being constantly sought.

Some neurodegenerative diseases also include an inflammatory component such as multiple sclerosis which traditionally was considered as inflammatory mediated demyelinating diseases but, in fact, is a neurodegenerative disease in which axonal damage, neuronal death and atrophy of the CNS are the principal causes of irreversible neurological disability in patients. Thus, multiple sclerosis can be considered as a neurodegenerative disease but also as a neuroinflammatory disease or autoimmune disease.

Cerebrovascular diseases are a group of brain dysfunctions related to disease of the blood vessels supplying the brain. There are four types: stroke, transient ischemic attack (TIA), subarachnoid hemorrhage and vascular dementia.

Epilepsy is an unpredictable, serious and potentially fatal disorder of the nervous system. About 50 million people worldwide have epilepsy.

Dementia is a clinical syndrome characterized by deficits in multiple areas of cognition that cannot be explained by normal aging, a noticeable decline in function, and an absence of delirium. In addition, neuropsychiatric symptoms and focal neurological findings are usually present. Dementia is further classified based on etiology Alzheimer's disease (AD) is the most common cause of dementia, followed by mixed AD and vascular dementia, Lewy body dementia (DLB), and fronto-temporal dementia.

Alzheimer's disease is a particular problem. According to the National Institute on Aging, AD is a complex, and it is unlikely that any one drug will successfully treat it. Current approaches focus on helping people maintain mental function, manage behavioral symptom, and slow down the symptoms of disease. Several prescription drugs are currently approved by the U.S. Food and Drug Administration to treat people who have been diagnosed with AD. Most medicines work best for people in the early or middle stages of Alzheimer's. For example, they can slow down some symptoms, such as memory loss, for a time. According to the National Institute on Aging, “It is important to understand that none of these medications stops the disease itself.”

Consequently, it is understood that there is an urgent need for new treatments and preventative treatments for CNS disorders, especially dementia and most especially Alzheimer's disease.

### BRIEF SUMMARY

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this introduction, which is included for purposes of illustration only and not restriction.

The inventions described and claimed herein involve CNS disorders and their treatment with an agent capable of elevating or otherwise normalizing elevated or depressed copper values and an agent capable of elevating or normalizing depressed vitamin B₅ levels. Agents capable of elevating or normalizing copper values include copper chelator agents and agents capable of elevating or normalizing vitamin B₅ levels are vitamin B₅ agents. They are administered separately or together in combination. Preferred copper chelator agents are copper (II) chelators.

A preferred vitamin B₅ agent is vitamin B₅.

The present invention is based, in part, on new doses and dosage forms for treatments aimed at normalizing copper and vitamin B₅ levels that are useful, for example, in treating and preventing macrovascular, microvascular and/or toxic/metabolic diseases of the kind referenced herein and in tissue repair processes where copper levels are abnormal and vitamin B₅ levels are depressed. This is irrespective of the glucose metabolism of the subject and irrespective of whether or not fructosamine oxidase is involved in any such disease. The invention also relates to doses and dosage forms of treatments relating to the cardiovascular accumulation of redox-active transition metal ions in diabetes.

In one aspect of the invention there is provided a method for treating or preventing disorders of the central nervous system comprising administering to a mammal in need thereof, separately or in combination, an agent capable or normalizing CNS copper levels and an agent capable of increasing or normalizing vitamin B₅.

In one aspect, the invention comprises administering to a mammal in need thereof, separately or in combination (1) a therapeutically effective amount of a pantothenic acid (also known as Vitamin B₅) agent, including Vitamin B₅ itself, as well as phosphopantatheine, panthenol, or co-Enzyme A or a combination thereof or pharmaceutically acceptable salts thereof, and (2) a therapeutically effective amount of a copper chelating agent or a pharmaceutically acceptable salt thereof.

This invention concerns methods of treatment, prevention or amelioration of a central nervous system disorder in a mammal (hereafter “treating”), including, for example, a human being, with a therapeutically effective amount of a copper chelating agent such as triethylenetetramine and pharmaceutically acceptable salts thereof (such as the dihydrochloride or disuccinate salts) and a therapeutically effective amount of pantothenic acid (also known as Vitamin B₅) or another Vitamin B₅ agent or a pharmaceutically acceptable salt thereof.

In another aspect of the invention the method uses a composition comprising a pharmaceutically acceptable carrier and a therapeutically effective amount of a copper chelating agent, e.g., succinic acid addition salt of triethylenetetramine. In another aspect, the method uses a composition comprising a pharmaceutically acceptable carrier and a therapeutically effective amount of a vitamin B₅ agent, e.g., vitamin B₅ itself. In one aspect the copper chelating agent, e.g., a succinic acid addition salt of triethylenetetramine, and the vitamin B₅ agent, e.g., vitamin B₅, are administered to a subject as separate compositions at the same time or at different times. In another, the copper chelating agent and the vitamin B₅ agent are administered to a subject as composition containing both agents. Thus, for example, in one embodiment, a succinic acid addition salt of triethylenetetramine, e.g., triethylenetetramine disuccinate, and the vitamin B₅ are administered to a subject as a composition containing both agents.

In one preferred aspect of the invention the disorder of the central nervous system to be treated is Alzheimer's disease, and in a preferred embodiment, Alzheimer's disease is treated with the copper chelating agent and vitamin B₅ agent combination, administered as separate compounds or in combination, preferably orally by a capsule(s) containing one or both agents, respectively. In one embodiment, the CNS patient, for example, the Alzheimer's disease patient, does not have Wilson's disease.

In another aspect of the invention the disorder of the central nervous system is chosen from the group consisting of multiple sclerosis, motor neuron disease, Lewy body dementia, vascular dementia, epilepsy, schizophrenia, schizoaffective disorders, schizophreniform disorders, delusional syndromes and other psychotic conditions related and not related to taking psychoactive substances, affective disorder, bipolar disorder, mania, depression, anxiety disorders of various etiology, stress reactions, consciousness disorders, coma, delirium of alcoholic or other etiology, aggression, psychomotor agitation and other conduct disorders, sleep disorders of various etiology, withdrawal syndromes of various etiology, addiction, pain syndromes of various etiology, intoxication with psychoactive substances, cerebral circulatory disorders of various etiology, psychosomatic disorders of various etiology, conversion disorders, dissociative disorders, urination disorders, autism and other developmental disorders, including nocturia, stuttering, tics, cognitive disorders of various types, including Alzheimer's disease, Parkinson disease, psychopathological symptoms and neurological disorders in the course of other diseases of the central and peripheral nervous systems.

Copper chelators useful in the invention are described herein, and include any therapeutically effective copper chelator, whether now known or later developed. Preferred copper chelating agents are chelators of copper (II) (sometimes referred to as copper″, or Cu2+, which is the cupric ion). Preferred copper (II) chelators are triethylenetetramine (trientine) and pharmaceutically acceptable salts thereof. Preferred triethylenetetramine salts are dihydrochloride and disuccinate salts. The disuccinate is salt is most preferred.

Thus, in one aspect of the invention the copper chelating agent is triethylenetetramine or a pharmaceutically acceptable salt thereof, as noted. In another related aspect of the invention the pharmaceutically acceptable salt of triethylenetetramine is the dihydrochloride salt. In another related aspect of the invention the pharmaceutically acceptable salt of triethylenetetramine is the disuccinate salt.

In other aspects of the invention, the pharmaceutically acceptable salt is a polymorph of triethylenetetramine disuccinate.

In another aspect of the invention the pharmaceutically acceptable salt is a Form I polymorph of triethylenetetramine dihydrochloride. In another aspect of the invention the pharmaceutically acceptable salt is a Form II polymorph of triethylenetetramine dihydrochloride. Other useful polymorphs are described herein.

In another aspect of the invention the copper chelating agent is chosen from the group consisting of triethylenetetramine (trientine), trientine hydrochloride, ethylenediaminetetraacetic acid (EDTA), diethylenetriaminetetraacetic acid (DPTA), 2,2,2 tetramine tetrahydrochloride (TETA), 2,3,2 tetramine tetrahydrochloride, D-penicillamine (DPA), 1,4,8,11 tetraazacyclotretradecane (Cyclam), 5,7,7′,12,14,14′ hexamethyl-1,4,8,11 tetraazacyclotretradecane (Cyclam S), Sodium 2,3 dimercaptopropane-1-sulfonate (DMPS), N-acetylpenicillamine (NAPA), D-Penicillamine (PA),′ Desferroxamine, 2,3-dimercaptopropanol (BAL), 2,3-dimercaptosuccinic acid (DMSA), trithiomolybdate, 3-7-Diazanonan-1,9-diamin (BE 6184), 1,4,8,11-tetraazacyclotetradecane-1,4,8,11-tetraacetic acid, 1,4,8,11-tetraazabicyclo [6.6.2]hexadecane, 4,11-bis(N,N-diethyl-amidomethyl)-1,4,8,11-tetraazabicyclo [6.6.2]hexadecane, 4,11-bis(amidoethyl)-1,4,8,11-tetraazabicyclo [6.6.2]hexadecane, melatonin, clioquinol, cuprizone, N,N′-diethyldithiocarbamate, zinc acetate, zinc salts, bathocuproinedisulfonic acid, bathocuprinedisulfonate, neocuproine (2,9-dimethyl-1,10-phenanthroline), tetrathiomolybdate, trimetazidine, triethylene tetramine tetrahydrochloride, 2,3,2-tetraamine, pyridine-2,6-bis(thiocarboxylic acid) or pyrrolidine dithiocarbamate, tetraethylenepentamine, N,N,N′,N-tetrakis(2-pyridylemethyl) ethylenediamine, 1,4,7,11-tetraazaundecane tetrahydrochloride, tetraethylenepentamine pentahydrochloride, D-Penicillamine (DPA), 1,10-orthophenanthroline, 3,4-Dihydroxybenzoic acid, 2,2′-bicinchinonic acid, diamsar, 3,4′,5, trihydroxystilbene (resveratrol), mercaptodextran, o-phenanthroline, disulfiram (antabuse), sar, calcium trisodium diethylenetriaminepentaacetate (salt of cpd above), and methimazole (1-methyl-2-thiolimidazole) and pharmaceutically acceptable salts thereof. In another aspect, the copper chelator agent is another therapeutically useful copper (II) chelator that is now known but not listed herein, or is a later developed therapeutically useful copper (II) chelator.

In another aspect of the invention the method comprises administering the copper chelating agent to a mammal in an amount ranging from about 9 mg/kg to about 200 mg/kg per day. In another aspect of the invention the method comprises orally administering to a mammal a copper chelating agent in an amount ranging from about 1.2 to about 2.4 grams per day. Other doses and dose ranges are described below. In one aspect of the invention the total daily dose administered ranges from 50 mg to 2500 mg of the succinic acid addition salt of triethylenetetramine.

In another aspect of the invention pantothenic acid (also known as Vitamin B₅), or another Vitamin B₅ agent, e.g., phosphopantatheine, panthenol, or co-Enzyme A or a combination thereof or pharmaceutically acceptable salts thereof is administered to a patient in need thereof with depressed vitamin B₅ levels in the range of about 1 mg to about 1000 mg preferably between about 10 mg and about 250 mg and more preferably between about 10 mg and about 100 mg.

The copper chelating agent and Vitamin B₅ agent doses may be provided, alone or together, in the form of pills, tablets or capsules for oral administration, the latter being preferred.

Thus, the invention also related to pharmaceutical compositions for the treatment, prevention or amelioration of a central nervous system disorder in a mammal (hereafter “treating”), including, for example, a human being, with a therapeutically effective amount of a copper chelating agent and a therapeutically effective amount of a pantothenic acid (also known as Vitamin B₅) agent or a pharmaceutically acceptable salt thereof. In one preferred embodiment, the subject is a human treated for Alzheimer's disease.

In another aspect of the invention the copper chelator, preferably the hydrochloric or succinic acid addition salt of triethylenetetramine, is in any of the compositions or formulations described herein and further comprises a therapeutically effective amount of pantothenic acid (also known as Vitamin B₅), phosphopantatheine, panthenol, or co-Enzyme A or a combination thereof or pharmaceutically acceptable salts thereof and a pharmaceutically acceptable carrier.

The copper chelating agent and the vitamin B₅ agent may administered to a subject separately or in combination. Thus, in another aspect of the invention the method uses a composition comprising a pharmaceutically acceptable carrier and a therapeutically effective amount of a copper chelating agent, e.g., succinic acid addition salt of triethylenetetramine. In another aspect, the method uses a composition comprising a pharmaceutically acceptable carrier and a therapeutically effective amount of a vitamin B₅ agent, e.g., vitamin B₅ itself. In one aspect the copper chelating agent, e.g., a succinic acid addition salt of triethylenetetramine, and the vitamin B₅ agent, e.g., vitamin B₅, are administered to a subject as separate compositions at the same time or at different times. In another, the copper chelating agent and the vitamin B₅ agent are administered to a subject as composition containing both agents. Thus, for example, in one embodiment, a succinic acid addition salt of triethylenetetramine, e.g., triethylenetetramine disuccinate, and the vitamin B₅ are administered to a subject as a composition containing both agents. In a preferred embodiment, the subject is a human and is treated for Alzheimer's disease with triethylenetetramine disuccinate and vitamin B₅, administered separately or together.

In light of the above, it will be understood that in one aspect of the invention the copper chelator, preferably the hydrochloric or succinic acid addition salt of triethylenetetramine, for example, composition and/or the vitamin B₅ agent, preferably Vitamin B₅, for example, are in a form suitable for oral administration. In another aspect of the invention the copper chelator and/or the vitamin B₅ agent, preferably the hydrochloric or succinic acid addition salt of triethylenetetramine and vitamin B₅, are in a form suitable for oral administration namely a capsule. In another aspect of the invention the copper chelator and/or the vitamin B₅ agent, preferably the hydrochloric or succinic acid addition salt of triethylenetetramine and vitamin B₅, are in a form suitable for oral administration namely a tablet. In another aspect are of the invention the copper chelator and the vitamin B₅ agent in a form suitable for oral administration namely an enteric-coated tablet or a layered tablet.

In another aspect of the invention the copper chelator and/or the vitamin B₅ agent, preferably the hydrochloric or succinic acid addition salt of triethylenetetramine and vitamin B₅, are in a form suitable for oral administration is a sustained release preparation, a delayed release preparation, a slow release preparation, a controlled release preparation, or an extended release preparation. In one aspect of the invention the copper chelator and/or the vitamin B₅ agent, preferably the hydrochloric or succinic acid addition salt of triethylenetetramine and vitamin B₅, are in forms suitable for transdermal administration, for transmucosal administration, for parenteral administration, for injection, or for administration as a suppository.

In another embodiment of the invention, an article of manufacture, or “kit”, containing materials useful for treating the diseases and disorders described herein is provided. The kit comprises a container comprising a copper chelating agent (e.g., succinic acid addition salt of triethylenetetramine) and/or a vitamin B₅ agent (e.g., vitamin B₅). The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, e.g., bottles, vials, syringes, blister pack, etc. The container may be formed from a variety of materials such as glass or plastic. The container may hold a copper chelating agent and/or a vitamin B₅ agent, e.g. a succinic acid addition salt of triethylenetetramine and/or vitamin B₅, or a formulation thereof which is effective for treating the condition and may have a sterile access port (e.g., the container may be an intravenous solution bag or a vial having a stopper pierceable by a hypodermic injection needle). The container may also be a package containing a composition in the form of a tablet or capsule, the latter being preferred, where the copper chelating agent and a vitamin B₅ agent are provided as separate compositions or together in combination in a single composition, e.g., in combined tablet or capsule. The label or package insert indicates that the composition(s) is/are used for treating a CNS condition of choice, such Alzheimer's disease, or more of the other disorders described herein.

### DETAILED DESCRIPTION OF THE INVENTION

The inventions described and claimed herein involve CNS disorders and their treatment with an agent capable of elevating or otherwise normalizing elevated or depressed copper values and an agent capable of elevating or normalizing depressed vitamin B₅ levels. Agents capable of elevating or normalizing copper values include copper chelator agents and agents capable of elevating or normalizing vitamin B₅ levels are vitamin B₅ agents. They are administered separately or together in combination. Preferred copper chelator agents are copper (II) chelators. A preferred vitamin B₅ agent is vitamin B₅.

The present invention is based, in part, on new doses and dosage forms for treatments aimed at normalizing copper and vitamin B₅ levels that are useful, for example, in treating and preventing macrovascular, microvascular and/or toxic/metabolic diseases of the kind referenced herein and in tissue repair processes where copper levels are abnormal and vitamin B₅ levels are depressed. This is irrespective of the glucose metabolism of the subject and irrespective of whether or not fructosamine oxidase is involved in any such disease. The invention also relates to doses and dosage forms of treatments relating to the cardiovascular accumulation of redox-active transition metal ions in diabetes.

In one aspect of the invention there is provided a method for treating or preventing disorders of the central nervous system comprising administering to a mammal in need thereof, separately or in combination, an agent capable of normalizing CNS copper levels and an agent capable of increasing or normalizing vitamin B₅.

In one aspect, the invention comprises administering to a mammal in need thereof, separately or in combination (1) a therapeutically effective amount of a pantothenic acid (also known as Vitamin B₅) agent, including Vitamin B₅ itself, as well as phosphopantatheine, panthenol, or co-Enzyme A or a combination thereof or pharmaceutically acceptable salts thereof, and (2) a therapeutically effective amount of a copper chelating agent or a pharmaceutically acceptable salt thereof.

The inventions described and claimed herein involve CNS disorders and their treatment with methods and compositions comprising, consisting essentially of, or consisting of copper chelator agents and vitamin B₅ agents, administered alone or together.

The invention provides the use of a copper chelator agent in the treatment of or the manufacture of a medicament for use in the treatment of one or more diseases, disorders and conditions described or referred to herein. The medicament will comprise, consist essentially of, or consist of a copper chelator agent. In one embodiment, the medicament will comprise, consist essentially of, or consist of a succinic acid addition salt of triethylenetetramine. In one embodiment, the medicament will comprise, consist essentially of, or consist of a vitamin B₅ agent. In one embodiment, the medicament will comprise, consist essentially of, or consist of vitamin B₅. In one embodiment, the medicament will comprise, consist essentially of, or consist of a copper chelator agent and a vitamin B₅ agent. In one embodiment, the medicament will comprise, consist essentially of, or consist of a succinic acid addition salt of triethylenetetramine and vitamin B₅. The term “comprising,” which is synonymous with “including,” “containing,” or “characterized by,” is inclusive or open-ended and does not exclude additional, unrecited elements or ingredients from the medicament (or steps, in the case of a method). The phrase “consisting of” excludes any element, step, or ingredient not specified in the medicament (or steps, in the case of a method). The phrase “consisting essentially of” refers to the specified materials and those that do not materially affect the basic and novel characteristics of the medicament (or steps, in the case of a method). The basic and novel characteristics of the inventions are described throughout the specification, and include the ability of medicaments and methods of the invention to modulate vitamin B₅ and copper (particularly copper (II)) levels in a subject. Material changes in the basic and novel characteristics of the inventions, including the medicaments and methods described herein, include an unwanted or clinically undesirable, detrimental, disadvantageous or adverse diminution of vitamin B₅ and/or attenuation of copper normalization in a subject. In one embodiment, the medicament will comprise, consist essentially of, or consist of triethylenetetramine disuccinate and vitamin B₅ and a pharmaceutically acceptable carrier. In one embodiment, the methods of treatment will comprise, consist essentially of, or consist of the administration of triethylenetetramine disuccinate and vitamin B₅, separately or together, to a subject in need thereof.

In a preferred embodiment, the subject has, is suspected of having, is developing, is suspected of developing, or is expected to develop Alzheimer's disease. These subjected may be referred to herein as “Alzheimer's disease patients” or as “subjects or patients with Alzheimer's disease.” In one embodiment, the CNS patient, for example, the Alzheimer's disease patient, does not have Wilson's disease.

In one aspect, the present invention is an orally consumed fixed combination formulation of both a copper chelator agent and a vitamin B₅ agent in one tablet that is expected to have the same Area Under Curve as two active ingredients taken together individually orally, and pharmaceutically acceptable additives suitable for the preparation. In one embodiment the formulation is a fixed combination of triethylenetetramine and vitamin B₅. In preferred embodiments of this invention, the triethylenetetramine in the fixed combination is in the form of triethylenetetramine dihydrochloride or triethylenetetramine disuccinate and the pharmaceutically acceptable additives are selected from diluents, disintegrants, glidants, lubricants, colorants and combinations thereof. The vitamin B₅ agent may also be an agent that is metabolized to vitamin B₅ in vivo.

The copper chelating agent may be a prodrug that releases the copper chelator agent in vivo.

In one embodiment, the fixed combination formulation is a long-acting or slow-release formulation. For separate or combined administration, the formulation may be prepared to provide for rapid or slow release; immediate, delayed, timed, or sustained release; or a combination thereof. Formulations may be in the form of liquids, solutions, suspensions, emulsions, elixirs, syrups, electuaries, drops (including but not limited to eye drops), tablets, granules, powders, lozenges, pastilles, capsules, gels, ointments, creams, lotions, oils, foams, sprays, mists, or aerosols. Capsules are presently preferred.

Preferred copper chelators are copper (II) chelators. A preferred copper (II) chelator is a succinic acid addition salt of triethylenetetramine, most preferably triethylenetetramine disuccinate.

Treatment of Alzheimer's disease patients using the methods and pharmaceutical compositions described and claimed herein is a preferred therapy.

### Definitions

As used herein, the term “CNS disorder” refers to any disease, disorder or condition of the central nervous system where modulation of Vitamin B₅ and copper, particularly copper (II), may be of benefit, and includes any disease, disorder or condition in which depressed or reduced Vitamin B₅ and depressed, reduced or increased copper, particularly copper (II), and particularly depressed or reduced copper (II), may be implicated in the onset, progression, or persistence of the disease, disorder or condition.

CNS disorders include Alzheimer's disease. CNS disorders include multiple sclerosis, Lewy body dementia, vascular dementia, epilepsy, schizophrenia, schizoaffective disorders, schizophreniform disorders, motor neuron disease, delusional syndromes and other psychotic conditions related and not related to taking psychoactive substances, affective disorder, bipolar disorder, mania, depression, anxiety disorders of various etiology, stress reactions, consciousness disorders, coma, delirium of alcoholic or other etiology, aggression, psychomotor agitation and other conduct disorders, sleep disorders of various etiology, withdrawal syndromes of various etiology, addiction, pain syndromes of various etiology, intoxication with psychoactive substances, cerebral circulatory disorders of various etiology, psychosomatic disorders of various etiology, conversion disorders, dissociative disorders, urination disorders, autism and other developmental disorders, including nocturia, stuttering, tics, cognitive disorders of various types, including Alzheimer's disease, Parkinson disease, psychopathological symptoms and neurological disorders in the course of other diseases of the central and peripheral nervous systems.

The invention also includes treatment of cerebrovascular diseases, i.e., the group of brain dysfunctions related to disease of the blood vessels supplying the brain. There are four types that may be treated with the methods and compositions of the invention: stroke (particularly hemorrhagic), transient ischemic attack (TIA), subarachnoid hemorrhage and vascular dementia.

By “in combination with” or “in conjunction with”, is meant that an amount of the copper chelating agent is administered anywhere from simultaneously to about 1 hour or more, e.g., about 2 hours or more, about 3 hours or more, about 4 hours or more, about 5 hours or more, about 6 hours or more, about 7 hours or more, about 8 hours or more, about 9 hours or more, about 10 hours or more, about 11 hours or more, or about 12 hours or more, about 13 hours or more, about 14 hours or more, about 15 hours, about 16 hours or more, about 17 hours or more, about 18 hours or more, about 19 hours or more, about 20 hours or more, about 21 hours or more, about 22 hours or more, about 23 hours or more, about or 24 hours or more, prior to, or after, the vitamin B₅ agent. That is to say, in certain embodiments, the copper chelating agent and vitamin B₅ agent are administered sequentially, e.g., where the copper chelating agent is administered before or after the vitamin B₅ agent. In other embodiments, the copper chelating agent and vitamin B₅ agent are administered simultaneously, e.g., where the copper chelating agent and vitamin B₅ agent are administered at the same time as two separate formulations, or are combined into a single composition that is administered to the subject. Regardless of whether the copper chelating agent and vitamin B₅ agent are administered sequentially or simultaneously, as illustrated above, or any effective variation thereof, the agents are considered to be administered together or in combination for purposes of the present invention. Routes of administration of the two agents can vary, where representative routes of administration are described in greater detail below. In the invention, copper chelating agents are provided in combination with or in conjunction with vitamin B₅ agents.

As used herein the terms “subjecting the patient” or “administering to” includes any active or passive mode of ensuring the in vivo presence of the active compound(s) or metabolite(s) irrespective of whether one or more dosage to the mammal, patient or person is involved. Preferably the mode of administration is oral. However, all other modes of administration (particularly parenteral, e.g., intravenous, intramuscular, etc.) are also contemplated.

The term “treating CNS disorders” or the like, including diseases and conditions, may refer to preventing, slowing, reducing, decreasing, stopping and/or reversing the CNS disorder, disease or condition, such as, for example, one or more symptoms thereof.

“Treating Alzheimer's disease refers to preventing, slowing, reducing, decreasing, stopping and/or reversing the disease, including, for example, one or more symptoms thereof. Alzheimer's disease symptoms addressed by the methods and compositions of the invention include improvement in one or more of memory loss, difficulty with remembering or organizing thoughts, difficulty concentrating, dementia, cognitive or behavioral impairment involving a minimum of two of the following domains: (1) impaired ability to acquire and remember new information (symptoms include repetitive questions or conversations, misplacing personal belongings, forgetting events or appointments, getting lost on a familiar route); (2) impaired reasoning and handling of complex tasks, poor judgment; (3) impaired visuospatial abilities; (4) impaired language functions (speaking, reading, writing); (5) changes in personality, behavior, or comportment (symptoms include: uncharacteristic mood fluctuations such as agitation, impaired motivation, initiative, apathy, loss of drive, social withdrawal, decreased interest in previous activities, loss of empathy, compulsive or obsessive behaviors, socially unacceptable behaviors).

The term “preventing” means preventing in whole or in part, or ameliorating or controlling.

As used herein, “effective amount” refers to an amount effective, at dosages and for periods of time necessary, to achieve the desired therapeutic or prophylactic result. For example, and not by way of limitation, an “effective amount” can refer to an amount of a compound or composition, disclosed herein, that is able to treat the signs and/or symptoms of a disease, disorder or condition, e.g., Alzheimer's disease.

As used herein, “therapeutically effective amount” of a substance/molecule of the invention, agonist or antagonist may vary according to factors such as the disease state, age, sex, and weight of the individual, and the ability of the substance/molecule, agonist or antagonist to elicit a desired response in the individual. A therapeutically effective amount is preferably also one in which any toxic or detrimental effects of the substance/molecule, agonist or antagonist may be outweighed by the therapeutically beneficial effects. A “therapeutically effective amount” is typically a predetermined amount of an agent that will or is calculated to achieve a desired response, for example, a therapeutic or preventative or ameliorating response, for example, a biological or medical response of a tissue, system, animal or human that is sought, for example, by a researcher, veterinarian, medical doctor, or other clinician.

As used herein, “prophylactically effective amount” refers to an amount effective, at dosages and for periods of time necessary, to achieve a desired prophylactic result. Typically but not necessarily, since a prophylactic dose is used in subjects prior to or at an earlier stage of a disease, disorder or condition, the prophylactically effective amount may be less than the therapeutically effective amount.

By “pharmaceutically acceptable” it is meant, for example, a carrier, diluent or excipient that is compatible with the other ingredients of the formulation and generally safe for administration to a recipient thereof or that does not cause an undesired adverse physical reaction upon administration.

As used herein, “mammal” has its usual meaning and includes primates (e.g., humans and nonhumans primates), experimental animals (e.g., rodents such as mice and rats), farm animals (such as cows, hogs, sheep and horses), and domestic animals (such as dogs and cats).

As used herein, the terms “treatment” or “treating” of a condition, disorder, and/or a disease in a mammal, means, where the context allows, (i) preventing the condition or disease, that is, avoiding one or more clinical symptoms of the disease; (ii) inhibiting the condition or disease, that is, arresting the development or progression of one or more clinical symptoms; and/or (iii) relieving the condition or disease, that is, causing the regression of one or more clinical symptoms. Thus, “treatment” (and grammatical variations thereof such as “treat” or “treating”) normally refers to clinical intervention in an attempt to alter the natural course of the individual, tissue or cell being treated, and can be performed either for prophylaxis or during the course of clinical pathology. Desirable effects of treatment include, but are not limited to, preventing occurrence or recurrence of a disease, disorder or condition, alleviation of signs or symptoms, diminishment of any direct or indirect pathological consequences of the disease, decreasing the rate of disease progression, amelioration or palliation of the disease state, and remission or improved prognosis. In some embodiments, compounds, methods and compositions of the invention can be used to delay development of a disease, disorder or condition, or to slow the progression of a disease, disorder or condition. The term does not necessarily imply that a subject is treated until total recovery. Accordingly, “treatment” includes reducing, alleviating or ameliorating the symptoms or severity of a particular disease, disorder or condition or preventing or otherwise reducing the risk of developing a particular disease, disorder or condition. It may also include maintaining or promoting a complete or partial state of remission of a condition.

As used herein “associated with” simply means both circumstances exist and should not be interpreted as meaning one necessarily is causally linked to the other.

The term “chelatable copper” includes copper in any of its chelatable forms including different oxygen states such as copper (II). Accordingly, the term “copper values” (for example, elemental, salts, etc.) means copper in any appropriate form in the body available for such chelation (for example, in extracellular tissue and possibly bound to cell exteriors and/or collagen as opposed to intracellular tissue) and/or capable of being reduced by other means (for example, zinc administration). Methods and compositions of the invention are used to bind chelatable copper, preferably chelatable copper (II) and to normalize copper values or return copper values toward normal levels.

The term “pharmaceutical formulation” refers to a preparation which is in such form as to permit the biological activity of an active ingredient contained therein to be effective, and which does not contain additional components that are unacceptably toxic to a subject to which the formulation would be administered. Pharmaceutical formulations of the invention comprise a copper chelating agent and/or a vitamin B₅ agent.

“Copper chelating agents” bind or modify copper, preferably selectively binding or modifying copper (II) values and are used to normalize blood and/or tissue copper levels and to prevent unwanted copper accumulation. Copper chelating agents include prodrugs thereof. Other agents that normalize copper values, and other agents that selectively bind to or modify copper (II), whether now known or later developed, are included within this definition.

“Vitamin B₅ agents” include Vitamin B₅ (also known as pantothenic acid), as well as phosphopantatheine, panthenol, and co-Enzyme A and combinations thereof or pharmaceutically acceptable salts thereof. Vitamin B₅ agents include any pharmaceutically acceptable compounds that metabolize to vitamin B₅ in vivo, whether now known or later developed, following administration to a subject, for example, an Alzheimer's disease patient. Other agents that normalize vitamin B₅ levels are included within this definition.

As used herein, the term “subject” or the like, including “individual,” and “patient”, all of which may be used interchangeably herein, refers to any mammal, including humans, domestic and farm animals, and zoo, wild animal park, sports, or pet animals, such as dogs, horses, cats, sheep, pigs, cows, etc. The preferred mammal herein is a human, including adults, children, and the elderly. Preferred sports animals are horses and dogs. Preferred pet animals are dogs and cats. The subject may be, for example, an aquatic park animal, such as a dolphin, whale, seal or walrus. In certain embodiments, the subject, individual or patient is a human.

Copper chelating agents and vitamin B₅ agents may be administered alone or in combination with one or more additional ingredients and may be formulated into pharmaceutical compositions including one or more pharmaceutically acceptable excipients, diluents and/or carriers.

A “pharmaceutically acceptable carrier,” as used herein, refers to an ingredient in a pharmaceutical formulation, other than an active ingredient, which can be safely administered to a subject. A pharmaceutically acceptable carrier includes, but is not limited to, a buffer, excipient, stabilizer, or preservative. Pharmaceutically acceptable diluents, carriers and/or excipients include substances that are useful in preparing a pharmaceutical composition, may be co-administered with compounds described herein while allowing them to perform its intended functions, and are generally safe, non-toxic and neither biologically nor otherwise undesirable. Pharmaceutically acceptable diluents, carriers and/or excipients include those suitable for veterinary use as well as human pharmaceutical use. Suitable carriers and/or excipients will be readily appreciated by persons of ordinary skill in the art, having regard to the nature of compounds of the invention. However, by way of example, diluents, carriers and/or excipients include solutions, solvents, dispersion media, delay agents, polymeric and lipidic agents, microspheres, emulsions and the like. By way of further example, suitable liquid carriers, especially for injectable solutions, include water, aqueous saline solution, aqueous dextrose solution, and the like, with isotonic solutions being preferred for intravenous, intraspinal, and intracisternal administration and vehicles such as liposomes being also suitable for administration of the agents of the invention.

Compositions may take the form of any standard known dosage form including tablets, pills, capsules, semisolids, powders, sustained release formulation, solutions, suspensions, elixirs, aerosols, liquids for injection, gels, creams, transdermal delivery devices (for example, a transdermal patch), inserts such as CNS inserts, or any other appropriate compositions. Persons of ordinary skill in the art to which the invention relates will appreciate the most appropriate dosage form having regard to the nature of the condition to be treated and the active agent to be used without any undue experimentation. Various doses and dose ranges are described herein. It should be appreciated that one or more of the copper chelating agents and vitamin B₅ agents may be formulated into a single composition. In certain embodiments, preferred dosage forms include an injectable solution and an oral formulation.

In addition to standard diluents, carriers and/or excipients, a composition in accordance with the invention may be formulated with one or more additional constituents, or in such a manner, so as to enhance the activity or bioavailability of the copper chelating agents and/or vitamin B₅ agents, help protect the integrity or increase the half-life or shelf life thereof, enable slow release upon administration to a subject, or provide other desirable benefits, for example. For example, slow release vehicles include macromers, poly(ethylene glycol), hyaluronic acid, poly(vinylpyrrolidone), or a hydrogel. By way of further example, the compositions may also include preserving agents, solubilizing agents, stabilizing agents, wetting agents, emulsifying agents, sweetening agents, coloring agents, flavoring agents, coating agents, buffers and the like. Those of skill in the art to which the invention relates will readily identify further additives that may be desirable for a particular purpose.

Compounds of the invention may be administered by a sustained-release system. Suitable examples of sustained-release compositions include semi-permeable polymer matrices in the form of shaped articles, e.g., films, or microcapsules. Sustained-release matrices include polylactides (U.S. Pat. No. 3,773,919; EP 58,481), copolymers of L-glutamic acid and gamma-ethyl-L-glutamate, poly(2-hydroxyethyl methacrylate), ethylene vinyl acetate, or poly-D-(−)-3-hydroxybutyric acid (EP 133,988). Sustained-release compositions also include a liposomally entrapped compound. Liposomes containing copper chelating agents and/or vitamin B₅ agents may be prepared by known methods, including, for example, those described in: DE 3,218,121; EP 52,322; EP 36,676; EP 88,046; EP 143,949; EP 142,641; Japanese Pat. Appln. 83-118008; U.S. Pat. Nos. 4,485,045 and 4,544,545; and EP 102,324. Ordinarily, the liposomes are of the small (from or about 200 to 800 Angstroms) unilamellar type in which the lipid content is greater than about 30 mole percent cholesterol, the selected proportion being adjusted for the most efficacious therapy. Slow release delivery using PGLA nano- or microparticles, or in situ ion activated gelling systems may also be used, for example.

Additionally, it is contemplated that a pharmaceutical composition in accordance with the invention may be formulated with additional active ingredients or agents which may be of therapeutic or other benefit to a subject in particular instances. Persons of ordinary skill in the art to which the invention relates will be able to identify suitable additional active ingredients having regard to the description of the invention herein and nature of the disorder to be treated.

The compositions may be formulated in accordance with standard techniques as may be found in such standard references as Gennaro A R: Remington: The Science and Practice of Pharmacy, 20.sup.th ed., Lippincott, Williams & Wilkins, 2000, for example.

In certain embodiments, the invention provides a combination product comprising (a) copper chelating agents, for example copper (II) chelator (e.g., a succinic acid addition salt of triethylenetetramine, preferably triethylenetetramine disuccinate), and (b) one or more vitamin B₅ agents, e.g., vitamin B₅, wherein the components (a) and (b) are adapted for administration simultaneously or sequentially. In a particular embodiment of the invention, a combination product in accordance with the invention is used in a manner such that at least one of the components is administered while the other component is still having an effect on the subject being treated.

The copper chelating agents and/or vitamin B₅ agents may be contained in the same or one or more different containers and administered separately, or mixed together, in any combination, and administered concurrently. Preferably, they are combined in a capsule for oral administration.

Such combination products may be manufactured in accordance with the methods and principles provided herein and those known in the art. Also provided is combination product used in a method as herein described.

For separate or common administration, the formulation may be prepared to provide for rapid or slow release; immediate, delayed, timed, or sustained release; or a combination thereof. Formulations may be in the form of liquids, solutions, suspensions, emulsions, elixirs, syrups, electuaries, drops (including but not limited to eye drops), tablets, granules, powders, lozenges, pastilles, capsules, gels, ointments, creams, lotions, oils, foams, sprays, mists, or aerosols. As an additional embodiment, the pharmaceutical formulation can be contained within, delivered by, or attached to contact lenses that are placed on the eye.

### Articles of Manufacture/Kits

The invention also includes an article of manufacture, or “kit”, containing materials useful for treating the diseases and disorders described herein is provided. The kit comprises a container comprising a copper chelating agent (e.g., succinic acid addition salt of triethylenetetramine) and/or a vitamin B₅ agent (e.g., vitamin B₅). The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, e.g., bottles, vials, syringes, blister pack, etc. The container may be formed from a variety of materials such as glass or plastic. The container may hold a copper chelating agent and/or a vitamin B₅ agent, e.g. a succinic acid addition salt of triethylenetetramine and/or vitamin B₅, or a formulation thereof which is effective for treating the condition and may have a sterile access port (e.g., the container may be an intravenous solution bag or a vial having a stopper pierceable by a hypodermic injection needle). The container may also be a package containing a composition in the form of a tablet or capsule, the latter being preferred, where the copper chelating agent and a vitamin B₅ agent are provided as separate compositions or together in combination in a single composition, e.g., in combined tablet or capsule. The label or package insert indicates that the composition(s) is/are used for treating a CNS condition of choice, such Alzheimer's disease, or more of the other disorders described herein.

### Copper Chelators

Copper chelators useful in the invention are described herein, and include any therapeutically effective copper chelator, whether now known or later developed. Preferred copper chelating agents are chelators of copper (II) (sometimes referred to as copper″, or Cu2+, which is the cupric ion). Preferred copper (II) chelators are triethylenetetramine (trientine) and pharmaceutically acceptable salts thereof, including hydrochloride and succinate salts. Preferred triethylenetetramine salts are dihydrochloride and disuccinate salts. The disuccinate is salt is most preferred.

Thus, in one aspect of the invention the copper chelating agent is triethylenetetramine or a pharmaceutically acceptable salt thereof, as noted. In another related aspect of the invention the pharmaceutically acceptable salt of triethylenetetramine is the dihydrochloride salt. In another related aspect of the invention the pharmaceutically acceptable salt of triethylenetetramine is the disuccinate salt.

Some preferred chelators of copper values appropriate for mammalian administration for treatment of one or more of the conditions, disorders and/or diseases herein include, for example (where appropriate as a salt such as, for example, a suitable calcium sodium salt to avoid hypocalcemia): trientine (triene), trientine hydrochloride, ethylenediaminetetraacetic acid (EDTA), diethylenetriaminetetraacetic acid (DPTA), 2,2,2 tetramine tetrahydrochloride (TETA), 2,3,2 tetramine tetrahydrochloride, D-peniciflamine (DPA), 1,4,8,11 tetraazacyclotretradecane (Cyclam), 5,7,7′,12,14,14′hexamethyl-1,4,8, 11 tetraazacyclotretradecane (Cyclam S), Sodium 2,3 dimercaptopropane-1-sulfonate (DMPS), N-acetylpenicillamine (NAPA), D-Penicillamine (PA),′ Desferroxamine, 2,3-dimercaptopropanol (BAL), 2,3-dimercaptosuccinic acid (DMSA), trithiomolybdate, 3-7-Diazanonan-1,9-diamin (BE 6184), 1,4,8,11-tetraazacyclotetradecane-, 1,4,8,11-tetraacetic acid, 1,4,8,11-tetraazabicyclo [6.6.2]hexadecane, 4,11-bis(N,N-diethyl-amidomethyl)-1,4,8, 11-tetraazabicyclo [6.6.2]hexadeca-ne, 4,11-bis(amidoethyl)-1,4,8, 11-tetraazabicyclo [6.6.2]hexadecane, melatonin, clioquinol, cuprizone, N,N′-diethyldithiocarbamate, zinc acetate, zinc salts, bathocuproinedisulfonic acid, bathocuprinedisulfonate, neocuproine (2,9-dimethyl-1,10-phenanthroline), tetrathiomolybdate, trimetazidine, triethylene tetramine tetrahydrochloride, 2,3,2-tetraamine, pyridine-2,6-bis(thiocarboxylic acid) or pyrrolidine dithiocarbamate, tetraethylenepentamine, N,N,N′,N-tetrakis(2-pyridylemethyl)ethylenediamine, 1,4,7,11-tetraazaundecane tetrahydrochloride, tetraethylenepentamine pentahydrochloride, D-Penicillamine (DPA), 1,10-orthophenanthroline, 3,4-Dihydroxybenzoic acid, 2,2′-bicinchinonic acid, diamsar, 3,4′,5, trihydroxystilbene (resveratrol), mercaptodextran, o-phenanthroline, disulfuram(antabuse), sar, calcium trisodium diethylenetriaminepentaacetate (salt of cpd above), and methimazole (1-methyl-2-thiolimidazole).

In another aspect of the invention the pharmaceutically acceptable salt is a polymorph of triethylenetetramine disuccinate has a DSC extrapolated onset and peak melting temperatures of from between about 170° C. to about 190° C. In another aspect of the invention the pharmaceutically acceptable salt is a polymorph of triethylenetetramine disuccinate has a DSC extrapolated onset and peak melting temperature are 180.05 and 179.91° C., respectively. In another aspect of the invention the pharmaceutically acceptable salt is a polymorph of triethylenetetramine disuccinate has infrared peaks at wavenumbers at 3148, 1645, 1549, 1529, 1370, 1271, 1172, 1152, and 1033(±2 cm⁻¹).

In another aspect of the invention the pharmaceutically acceptable salt is the Form I polymorph of triethylenetetramine dihydrochloride and is characterized by a DSC extrapolated onset and peak melting temperatures of between about 111° C. to 132° C. In another aspect of the invention the pharmaceutically acceptable salt is the Form I polymorph of triethylenetetramine dihydrochloride and is characterized by DSC extrapolated onset and peak melting temperatures are 121.96 and 122.78° C., respectively. In another aspect of the invention the pharmaceutically acceptable salt is the Form I polymorph of triethylenetetramine dihydrochloride characterized by infrared peaks at wavenumbers 1043, 1116, 1300, 1328, 1557, 2833, 2895, 2902, and 3216(±2 cm⁻¹).

In another aspect of the invention the pharmaceutically acceptable salt is the Form II polymorph of triethylenetetramine dihydrochloride characterized by a DSC extrapolated onset and peak melting temperature of from between about 106° C. to about 126° C. In another aspect of the invention the pharmaceutically acceptable salt is the Form II polymorph of triethylenetetramine dihydrochloride characterized by a DSC extrapolated onset and peak melting temperatures are 116.16 and 116.76° C., respectively. In another aspect of the invention the pharmaceutically acceptable salt is the Form II polymorph of triethylenetetramine dihydrochloride characterized by infrared peaks at wave numbers 1039, 1116, 1352, 1519, 2954, 2986, 3276, and 3298 (±2 cm⁻¹).

In another aspect of the invention the pharmaceutically acceptable salt is a polymorph of a triethylenetetramine disuccinate wherein the polymorph is a crystal having the structure defined by the co-ordinates of Table 3B found in U.S. Pat. No. 8,067,641. In another aspect of the invention the pharmaceutically acceptable salt is a polymorph of triethylenetetramine disuccinate wherein the polymorph is a crystal having the structure defined by the co-ordinates of Table 3C found in U.S. Pat. No. 8,067,641.

### Doses, Amounts and Concentrations

The invention comprises administering the copper chelating agent to a mammal in an amount ranging from about 9 mg/kg to about 200 mg/kg per day. In another aspect of the invention the method comprises orally administering to a mammal a copper chelating agent in an amount ranging from about 1.2 to about 2.4 grams per day. Other doses and dose ranges are described below.

In one aspect of the invention the total daily dose administered ranges from 50 mg to 2500 mg of the succinic acid addition salt of triethylenetetramine.

In another aspect of the invention the composition comprises from 50 mg to 500 mg of the succinic acid addition salt of triethylenetetramine. In another aspect of the invention the composition comprises from 110 to 290 mg of the succinic acid addition salt of triethylenetetramine. In another aspect of the composition comprises from 130 to 270 mg of the succinic acid addition salt of triethylenetetramine. In another aspect of the invention the composition comprises from 140 to 260 mg of the succinic acid addition salt of triethylenetetramine. In another aspect of the invention the composition comprises from 180 to 220 mg of the succinic acid addition salt of triethylenetetramine. In another aspect of the invention the composition comprises from 50 mg to 100 mg of the succinic acid addition salt of triethylenetetramine. In another aspect of the invention the composition comprises an amount of the succinic acid addition salt of triethylenetetramine selected from the group consisting of 50 mg, 110 mg, about 130 mg, 140 mg, and 150 mg. In another aspect of the invention the composition comprises an amount of the succinic acid addition salt of triethylenetetramine selected from the group consisting of 1.2 mg, 10 mg, 12 mg, 20 mg, 30 mg, and 40 mg.

In a related aspect of the invention pantothenic acid (also known as Vitamin B₅), or another Vitamin B₅ agent, e.g., phosphopantatheine, panthenol, or co-Enzyme A or a combination thereof or pharmaceutically acceptable salts thereof is administered to a patient in need thereof with depressed vitamin B₅ levels in the range of about 1 mg to about 1000 mg preferably between about 10 mg and about 250 mg and more preferably between about 10 mg and about 100 mg.

### Manufacture

Copper chelator agents, including for example triethylenetetramine dihydrochloride or triethylenetetramine disuccinate, and vitamin B₅ agents, including vitamin B₅, suitable for use in the present invention can be purchased from commercial sources or can be prepared according to art known methods. Two-component slow release preparations of a copper chelator agent and a vitamin B₅ agent in tablets or capsules are preferred, most preferably in capsules.

Pharmaceutical grade vitamin B₅ and vitamin B₅ agents are available from a wide variety of sources known in the art, including, for example, in raw pharma grade and slow-release forms.

Copper chelator agents may be obtained from known manufacturing sources or synthesized using methods know in the art. Some copper chelators are manufactured using methods described in U.S. Pat. No. 9,556,123, which describes the synthesis of triethylenetetramines and useful intermediates in their production. U.S. Pat. No. 8,912,362 describes and claims isolated triethylenetetramine hydrochloride and dihydrochloride salts of varying purity, including 95% pure, 96% pure, 97% pure, 98% pure, 99% pure and 100% pure. It also claims isolated triethylenetetramine salts with a purity of greater than about 99% pure and less than 10 ppm of heavy metals.

U.S. Pat. No. 8,394,992 describes a useful process for preparing triethylenetetramine dihydrochloride, comprising: (a) reacting triethylenetetramine tetrahydrochloride with a base in a solvent to produce triethylenetetramine and chloride salt; (b) removing said chloride salt from solution (e.g., by precipitation or filtration); (c) reacting the triethylenetetramine with about 2 equivalents of concentrated hydrochloric acid to form triethylenetetramine dihydrochloride; and (d) adding an alcohol to the solution and precipitating triethylenetetramine dihydrochloride. Bases include sodium methoxide and sodium ethoxide. Solvents include ethanol, methanol and tert-butylmethylether. Alcohols include ethanol, methanol and isopropanol. Yields can be greater than 86% and up to 100%. The '992 patent also claims thermodynamic polymorph of crystalline triethylenetetramine dihydrochloride.

U.S. Pat. No. 8,067,641 describes the preparation of polymorphs of triethylenetetramine disuccinate, including various Form I and Form II polymorphs, as well as pharmaceutical compositions with substantially pure polymorphs.

### Pharmaceutical Preparations

Also provided are pharmaceutical preparations. As used herein, pharmaceutical preparations mean compositions that include a copper chelator agent, for example, triethylenetetramine dihydrochloride or triethylenetetramine disuccinate, and a vitamin B₅ agents, for example, vitamin B₅ (either alone or in the presence of one or more additional active or inactive agents) present in a pharmaceutically acceptable vehicle. The term “pharmaceutically acceptable” has the meaning set forth above and includes those vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal.

The choice of excipient will be determined in part by the active ingredient, as well as by the particular method used to administer the composition. Accordingly, there is a wide variety of suitable formulations of the pharmaceutical composition of the present invention.

In one aspect, the present disclosure provides pharmaceutical preparation wherein the active agent is a copper (II) chelator, e.g., triethylenetetramine dihydrochloride or triethylenetetramine disuccinate, and a vitamin B₅ agent, e.g., vitamin B₅ or a pharmaceutically acceptable salt thereof. The dosage form of the copper chelator agent in the methods of the present invention can be prepared by combining the copper chelator agent with one or more pharmaceutically acceptable diluents, carriers, adjuvants, and the like in a manner known to those skilled in the art of pharmaceutical formulation. The dosage form of the vitamin B₅ agent employed in the methods of the present invention can be prepared by combining the vitamin B₅ agent with one or more pharmaceutically acceptable diluents, carriers, adjuvants, and the like in a manner known to those skilled in the art of pharmaceutical formulation. In some cases, and preferably, the dosage form of the copper chelator agent and the dosage form of the vitamin B₅ agent are combined in a single composition, as noted.

Particular formulations of the invention are in a solid form.

Particular formulations of the invention are in the form of a transdermal patch.

### EXAMPLES

The inventions are related to and describes the methods relating to discoveries surrounding increased tissue copper and mechanisms leading to tissue damage and the beneficial effect of administration of copper chelating compounds in conjunction with pantothenic acid (vitamin B₅) or its metabolites (phosphopantatheine, panthenol, or co-Enzyme A or a combination thereof) in the treatment of CNS disorders especially Alzheimer' s disease.

The following examples are put forth so as to provide those of ordinary skill in the art with a complete disclosure and description of how to make and use the present invention, and are not intended to limit the scope of what the inventors regard as their invention, nor are they intended to represent that the experiments below are all or the only experiments.

Examples 1 and 2 describes animal models for evaluating and quantifying the effects of the inventions described and claimed herein.

Example 3 describes a clinical trial for safety and tolerability in which a composition comprising a copper chelator agent and a vitamin B₅ agent is studied in Alzheimer's volunteers. The results show the safety of co-administering a copper chelator agent and a vitamin B₅ agent. These results will demonstrate that the patient exposure to a copper chelator agent and a vitamin B₅ agent can be done safely.

Example 4 describes a clinical trial in which a composition comprising a copper chelator agent and a vitamin B₅ agent is studied in volunteer patients with active CNS disorder, namely Alzheimer's Disease. The results will demonstrate the safety and efficacy of co-administering a copper chelator agent and a vitamin B₅ agent.

### Example 1— Treatment in Transgenic Animal Model

Methods and compositions described and claimed herein are tested in animal models, for example, in any one of the well known in vivo models for Alzheimer's (e.g., The McGill-R-Thy1-APP transgenic rat animal model of the familial form of Alzheimer's disease) or other CNS disorders by administering therapeutically effective amounts of a copper chelator preferably hydrochloric acid addition salts of triethylenetetramine or the succinate addition salts of triethylenetetramine preferably between about 100 mg to about 1.5 g per day and therapeutically effective amounts of pantothenic acid (vitamin B₅) or its metabolites (phosphopantatheine, panthenol, or co-Enzyme A or a combination thereof or pharmaceutically acceptable salts thereof), preferably pantothenic acid, preferably in an amount of about 0.1 mg to about 1.5 g per day. The agents are administered in peanut butter, for example, after which animals evaluated for signs of cognitive disabilities and/or dementia characteristic of Alzheimer's.

### Example 2— Treatment in Non-Transgenic Memory Deficit Mouse Model

A non-transgenic memory deficit mouse model is created using methods known in the art by a single ICV injection of Aβ(1-42) peptide, in order to mimic the early stage of Alzheimer's disease and the key role of amyloid oligomers in AD. No memory deficit is observed in the control mice with the antisense Aβ(42-1) peptide. Following administration selected doses of triethylenetetramine disuccinate and vitamin B₅ in peanut butter (to create a dose response curve) to memory deficient and control animals the capacity of a triethylenetetramine disuccinate and vitamin B₅ to partially or fully reverse the deficit of episodic memory after three weeks of treatment by oral route on the non-transgenic amyloid-impaired mice. Clioquinol and memantine are used as comparators to validate this fast and efficient mouse model.

In sum, the present disclosure provides multidrug combinations wherein a first copper chelating agent drug which is triethylenetetramine disuccinate is co-administered with a second vitamin B₅ agent drug which is vitamin B₅. In one aspect, the multidrug combinations provided herein can be used to treat patients who suffer from or have been diagnosed with a diseases or disorder, or who experience symptoms for which they are in need of treatment, such as patients who have been diagnosed with a CNS disorder including but not limited to patients diagnosed with Alzheimer's disease. In one aspect, the multidrug combinations provided herein can be used to treat, reduce, or ameliorate the frequency and/or severity of symptoms associated with such diseases or disorders.

### Example 3— Human Safety and Tolerability Study

This Phase I protocol is a double-blind, randomized, placebo-controlled, dose-rising, group-comparison safety and tolerability study of 6 months duration with secondary efficacy endpoints. The study protocol and informed consent process are approved by the authorized human subjects committee at each participating institution. Capsules containing from 1, 1.5 and to about 2.5 grams of triethylenetetramine disuccinate and capsules or tablets containing from 100, 200 and 500 mg of vitamin B₅ are administered daily to each subject volunteer. A placebo arm with standard of care treatment is included, for a total of four arms.

Eighty subjects meeting National Institute of Neurological and Communicative Disorders and Stroke—the Alzheimer's Disease and Related Disorders Association criteria for probable AD are enrolled. Enrollment criteria included Mini-Mental State Examination score between 12 and 26 and Clinical Dementia Rating (CDR) score of 1 (mild severity) or 2 (moderate severity) at the time of treatment randomization and administration of the first dose of study medication. Potential subjects with medical conditions that would influence cognition or likelihood of study completion (including heart failure) are excluded, as are those without a reliable caregiver willing to participate and comply with protocol responsibilities. Patients with diabetes mellitus requiring treatment with oral medications or insulin are also excluded. All subjects who provide consent are randomized and receive at least 1 dose of study medication. Medication compliance of more than 85% is required for continued participation and is assessed at each visit by pill counts.

Subjects may take prescribed cholinesterase inhibitor medications, provided they maintain a stable dose for 90 days prior to enrollment. Stable doses of antidepressant and antipsychotic drugs are also allowed if symptoms are adequately controlled. Memantine is approved for prescriptive use in the United States during this study. Memantine use is incorporated as a planned covariate in the outcome analyses.

For safety monitoring, baseline health status is evaluated by physical and neurological examinations, routine blood tests, and electrocardiogram; these are repeated at study conclusion. Every 3 months, physical and neurological examinations, complete blood cell count, blood glucose level, hemoglobin A_{1c} level, and hepatic function markers (alanine aminotransferase and aspartate aminotransferase levels) are obtained. Clinical adverse event monitoring is conducted according to FDA regulations. An independent safety monitoring committee systematically reviews adverse event reporting throughout the study.

Efficacy outcomes are evaluated to explore the potential magnitude of treatment effects on outcomes used in dementia clinical trials. The study is not designed to demonstrate statistically significant effects on these outcomes, as safety and tolerability are the primary endpoints. Outcome measures are collected every 3 months, with planned analyses for overall change over 6 months. If statistically significant differences are identified at 6 months, additional analyses of the 3-month data will be conducted to determine the time course of the treatment response. Clinical outcome measures are:
Clinical Dementia Rating sum of boxes (CDR-SB): This is an alternate scoring system, using the same data collection process as the CDR. In contrast to its standard scoring, which provides ordinal-level data, CDR-SB provides interval-scale data, which are amenable to the planned statistical analyses.
Alzheimer's Disease Assessment Scale Cognitive Score (ADAS-COG): This is a very sensitive 70-point psychometric scale for measuring cognitive function with an emphasis on memory, language, and praxis.
Neuropsychiatric Inventory: This was developed to assess behavioral disturbances occurring in patients with dementia. It rates severity and frequency of behavioral and psychiatric symptoms associated with dementia.
Alzheimer's Disease Functional Assessment and Change Score: This scale assesses changes in instrumental and basic activities of daily living over time.
Nurses' Observation Scale for Geriatric Patients: This is an easily administered scale designed to provide reliable assessments of multiple realms of function, namely memory, instrumental activities of daily living, self-care, mood, social behavior, and disturbing behavior. The instrument obtains caregiver ratings for each of 30 items on a 5-point scale according to frequency of occurrence. It provides a summary of the caregiver's interpretation of the impact of dementia on the subject's life.

Additionally, the Clinician's Interview-Based Impression—Plus is evaluated at baseline and study conclusion. This is a global rating derived through an independent, comprehensive interview between the subject and caregiver by a clinician who is barred from knowledge of all psychometric test scores. Using the results from baseline for reference, the clinician interviewed the subject and caregiver at the final visit to obtain an “Impression of Change.”

Given the small sample size, efficacy analyses are intended to be exploratory. t Tests and x² tests for independence are used, where appropriate, to test whether the 4 treatment groups (actives vs placebo) are similar or different in terms of age at baseline, education, sex, ethnicity (white vs minority), Mini-Mental State Examination score at baseline, presence of an APOE4 allele, and use of memantine during the study period.

The effects of triethylenetetramine disuccinate and vitamin B₅ on the clinical efficacy variables and on their rates of change over time are evaluated using multilevel models for repeated measures, except for Clinician's Interview-Based Impression—Plus Impression of Change for which only baseline and termination scores are obtained. Multilevel analysis, a statistical technique for analyzing data with nested variability, is also commonly referred to as “hierarchical linear modeling” or “mixed models.” It corrects for a lack of independence within clusters, such as several repeated observations within the same subject. Results are tabulated to evaluate safety and tolerability, and the efficacy of treatment of the Alzheimer's disease patient with triethylenetetramine disuccinate and vitamin B₅.

### Example 4— Human Efficacy Study with Triethylenetetramine Disuccinate and Vitamin B₅ in Patients with Alzheimer's Disease

Using the clinical outcome measures described in Example 3, an efficacy study is powered for a primary Alzheimer's efficacy endpoint and relevant secondary endpoints using selected doses selected from Example 3 in patient volunteers. Safety and tolerability and efficacy of treatment of the Alzheimer's disease patient with triethylenetetramine disuccinate and vitamin B₅ is evaluated after 12 months, with a pre-planned interim analysis at 6 months. This study is designed to measure the effects of co-administering triethylenetetramine disuccinate and vitamin B₅ in Alzheimer's disease patients, including safety and efficacy.

A multicenter, randomized, parallel, double-blind, placebo-controlled trial is carried out to evaluate the efficacy and tolerability of select daily doses triethylenetetramine disuccinate and vitamin B₅ administered following randomization of patients for 12 months in 285 patients with mild-to-moderate probable AD. The primary outcome measure is the Alzheimer's Disease Assessment Scale cognitive subscale (ADAS-cog); secondary outcome measures are the Clinical Global Impression of Change (CGIC) and the Progressive Deterioration Scale (PDS), and changes in functioning are assessed with the Alzheimer's Disease Cooperative Study— Activities of Daily Living (ADCS-ADL) scale, on which scores range from 0 to 78 and higher scores indicate better functioning. A mixed-model repeated-measures analysis is used and the efficacy of triethylenetetramine disuccinate and vitamin B₅ in Alzheimer's disease is quantified.

The effects of co-administration of a two-drug regimen is assessed in a clinical trial with Alzheimer's disease patients. It is expected that the discovery that CNS patients, particularly Alzheimer's disease patients, can be treated with the combination of a copper chelating agent and a vitamin B₅ agent will make it possible to treat a wider range of patients who have both depressed vitamin B₅ and reduced or increased copper, particularly copper (II), in order to normalize copper values and vitamin B₅ levels significant treatment effects but without adverse effects.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A pharmaceutical composition comprising a pharmaceutically acceptable carrier and therapeutically effective amounts of a copper (II) chelating agent and a vitamin B₅ agent.

2. The pharmaceutical composition of claim 1, wherein the copper chelator is triethylenetetramine or a pharmaceutically acceptable salt thereof.

3. The pharmaceutical composition of claim 1, wherein the copper chelating agent is a hydrochloric acid addition salt of triethylenetetramine or a succinic acid addition salt of triethylenetetramine.

4. The pharmaceutical composition of claim 1, wherein the vitamin B₅ agent is selected from the group consisting of vitamin B₅, phosphopantatheine, panthenol, or co-Enzyme A or a pharmaceutically acceptable salt thereof.

5. The pharmaceutical composition of claim 1, wherein the copper chelating agent is triethylenetetramine dihydrochloride and the vitamin B₅ agent is vitamin B₅.

6. The pharmaceutical composition of claim 1, wherein the copper chelating agent is triethylenetetramine disuccinate and the vitamin B₅ agent is vitamin B₅.

7. The pharmaceutical composition of claim 1, wherein the copper chelating agent is a polymorph of triethylenetetramine disuccinate.

8. The pharmaceutical composition of claim 1, wherein the copper chelating agent is a polymorph of triethylenetetramine dihydrochloride.

9. The pharmaceutical composition of claim 6, wherein the composition is formulated for timed-release or slow-release.

10. The pharmaceutical composition of claim 6, wherein the composition is formulated for oral administration.

11. The pharmaceutical composition of claim 10, wherein the composition is a capsule.

12. The pharmaceutical composition of claim 10, wherein the capsule contains from about 0.5 to about 2.5 grams of triethylenetetramine disuccinate and from about 100 to about 500 mg of vitamin B₅.

13. A method of treating a subject for Alzheimer's disease, the method comprising administering to said subject a therapeutically effective amount of a copper chelating agent and a therapeutically effective amount of a vitamin B₅ agent, wherein one or more Alzheimer's disease symptoms in said subject is improved.

14. The method of claim 13, wherein the copper chelating agent binds copper (II).

15. The method of claim 14, wherein the copper chelating agent that binds copper (II) is a succinic acid addition salt of triethylenetetramine.

16. The method of claim 15, wherein succinic acid addition salt of triethylenetetramine is triethylenetetramine disuccinate.

17. The method of claim 13, wherein the vitamin B₅ agent is selected from the group consisting of vitamin B₅, phosphopantatheine, panthenol, or co-Enzyme A or a pharmaceutically acceptable salt thereof.

18. The method of claim 13, wherein the vitamin B₅ agent is vitamin B₅.

19. The method of claim 12, wherein the copper chelating agent is administered in an amount ranging from about 9 mg/kg to about 200 mg/kg per day.

20. The method according to claim 12 comprising orally administering to said subject a copper chelating agent in an amount ranging from about 1.2 to about 2.4 grams per day.

21. The method of claim 12, wherein the copper chelating agent and the vitamin B₅ agent are administered in separate pharmaceutical compositions.

22. The method of claim 12, wherein the copper chelating agent and the vitamin B₅ agent are administered in a single pharmaceutical composition.

23. The method of claim 21 or 22, wherein the compositions are in a form suitable for oral administration.

24. The method of claim 23, wherein said form suitable for oral administration is a capsule.

25. The method of claim 23, wherein said form suitable for oral administration is a tablet.

26. The method according to claim 25, wherein said tablet is an enteric-coated tablet or a layered tablet.

27. The method of claim 23, wherein said form suitable for oral administration is a sustained release preparation, a delayed release preparation, a slow release preparation, a controlled release preparation, or an extended release preparation.

28. A pharmaceutical composition comprising therapeutically effective amounts of triethylenetetramine disuccinate and vitamin B₅.

29. A method of treating a subject for Alzheimer's disease comprising administering the composition of claim 28 to said subject.
